(19) United States
(12) Patent Application Publication
(10) Pub. No.: US 2010/0034759 A1
(43) Pub. Date: Feb. 11, 2010

(54) TANNING AID AND KIT FOR USE IN TANNING OF A HUMAN BODY SURFACE

(76) Inventor: Stella Ellzey, Huntsville, AL (US)

Correspondence Address: JAY M. SCHLOFF, 6960 Orchard Lake Road, Suite 315, West Bloomfield, MI, 48322, US

(21) Appl. No.: 12/507,148

(22) Filed: Jul. 22, 2009

Related U.S. Application Data
(60) Provisional application No. 61/087,336, filed on Aug. 8, 2008.

(51) Int. Cl. A61K 8/18; A61Q 17/04

(52) U.S. Cl. 424/59

(57) ABSTRACT

A tanning aid and a kit for use in tanning of a human body surface. The tanning aid comprises a tissue paper and a tanning composition. The tanning composition comprises an effective amount of at least one of a tanning lotion, a tanning oil and a bronzing agent. The tanning composition is impregnated in the tissue paper. A human body surface is wiped with the tissue paper impregnated with the tanning composition for applying the tanning composition to the human body surface.

### CROSS REFERENCE TO RELATED APPLICATIONS

The present disclosure claims priority under 35 United States Code, Section 119 on the U.S. Provisional Patent Application No. 61/087,336 filed on Aug. 8, 2008, the disclosure of which is incorporated by reference.

### FIELD OF THE DISCLOSURE

The present disclosure generally relates to personal care products, and, more particularly to a tanning aid and kit for use in tanning of a human body surface.

### BACKGROUND OF THE DISCLOSURE

Personal care products typically provide care in the form of nourishment to a desired portion of a human body for enhancing an attractiveness of the desired portion. For example, a personal care product, such as a facial cream, enhances a facial beauty of an individual, and, a personal care product such as a hair conditioner provides nourishment to hair of the individual. The personal care products may also be utilized for facilitating a cosmetic process. For example, a personal care product, such as a tanning oil or a tanning lotion (hereinafter collectively referred to as a tanning composition), facilitates tanning of a human body surface.

Tanning is a cosmetic process for imparting a tan by exposing the human body surface to Ultraviolet (UV) rays. The tanning of the human body surface may be carried out in an outdoor environment or an indoor environment. Conventionally, the tanning of the human body surface is carried out by exposing the human body surface to the UV rays of the sun in the outdoor environment. The tanning of the human body surface may also be performed by using the UV rays emitted by a tanning bed in the indoor environment. A tanning composition is, typically, applied to the human body surface prior to exposing the human body surface to the UV rays for facilitating the tanning of the human body surface. The tanning composition also protects the human body surface against harsh UV rays of the sun or the tanning bed.

Generally, the tanning composition is applied and spread over the human body surface with the help of human hands prior to exposing the human body surface to the UV rays. However, application of the tanning composition with the human hands may be time consuming and a cumbersome activity. Individuals who tan in tanning beds may have to make appointments at tanning centers, which may last only a few minutes and, as such, the individuals may have limited time to apply the tanning composition to the human body surface. Further, busy individuals that have limited time to apply the tanning composition may miss applying the tanning composition to one or more parts of the human body surface, or, may even skip the application of the tanning composition to the human body surface, altogether, thereby exposing the human body surface to harsh UV rays.

Furthermore, spreading the tanning composition with the help of human hands may lead to uneven application of the tanning composition over the human body surface. Such uneven application of the tanning composition over the human body surface may lead to development of uneven tan spots on the body. Furthermore, application of the tanning composition with the human hands may form clumps on the human body surface of an individual having abundance of body hair, thereby creating a mess.

Moreover, an individual may typically carry a bottle or a tube containing the tanning composition in a bag for applying the tanning composition on the human body surface. However, carrying such bottle or tube containing the tanning composition in the bag may be hazardous, as the bottle or the tube containing the tanning composition may develop a crack or may open (if improperly closed), thereby creating a mess in the bag.

Accordingly, there exists a need for enabling application of a tanning composition on a human body surface evenly and precluding clumps on the human body surface. Further, there exists a need for enabling application of the tanning composition on the human body surface in a timely and less cumbersome manner. Furthermore, there exists a need for precluding mess caused by leakage of tanning composition contained in bottles.

### SUMMARY OF THE DISCLOSURE

In view of the foregoing disadvantages inherent in the prior art, the general purpose of the present disclosure is to provide a tanning aid and a kit to include all the advantages of the prior art, and to overcome the drawbacks inherent therein.

Accordingly, an object of the present disclosure is to enable application of a tanning composition to a human body surface evenly and preclude clumps on the human body surface.

Another object of the present disclosure is to enable application of the tanning composition on the human body surface in a timely and less cumbersome manner.

Another object of the present disclosure to preclude messes caused by leakage of the tanning composition contained in bottles.

In light of the above objects, in one aspect of the present disclosure, a tanning aid is provided. The tanning aid comprises a tissue paper and a tanning composition. The tanning composition comprises an effective amount of at least one of a tanning lotion, a tanning oil and a bronzing agent. The tanning composition is impregnated in the tissue paper. A human body surface is wiped with the tissue paper impregnated with the tanning composition for applying the tanning composition to the human body surface.

In another aspect, the present disclosure provides a kit for use in tanning of a human body surface. The kit comprises a plurality of tanning aids and a tanning aid dispenser. Each tanning aid of the plurality of tanning aids comprises a tissue paper and a tanning composition. The tanning composition comprises an effective amount of at least one of a tanning lotion, a tanning oil and a bronzing agent. The tanning composition is impregnated in the tissue paper. The tanning aid dispenser is configured to store the plurality of tanning aids. The tanning aid dispenser is capable of dispensing a tanning aid of the plurality of tanning aids. The human body surface is wiped with the tissue paper impregnated with the tanning composition for applying the tanning composition to the human body surface.

Utilization of the tanning aid, i.e. the tissue paper impregnated with the tanning composition, for wiping the human body surface enables application of the tanning composition on the human body surface evenly and precludes clumps on the human body surface. Further, the tanning aid enables application of the tanning composition on the human body surface in a timely and less cumbersome manner. Furthermore, the tanning aid dispenser configured to store the plurality of tanning aids precludes the need to carry bottled tanning composition, thereby precluding messes caused by leakage in such bottles.

This together with other aspects of the present disclosure, along with the various features of novelty that characterize the present disclosure, is pointed out with particularity in the claims annexed hereto and form a part of this present disclosure. For a better understanding of the present disclosure, its operating advantages, and the specific objects attained by its uses, reference should be made to the accompanying drawing and descriptive matter in which there are illustrated exemplary embodiments of the present disclosure.

### BRIEF DESCRIPTION OF THE DRAWINGS

The advantages and features of the present disclosure will become better understood with reference to the following detailed description and claims taken in conjunction with the accompanying drawing, in which:

FIG. 1 illustrates a perspective view of a tanning aid, in accordance with an embodiment of the present disclosure;

FIG. 2 illustrates a perspective view of a kit for use in tanning of a human body surface, in accordance with an embodiment of the present disclosure;

FIG. 3 illustrates a user wiping his body surface with the tanning aid of FIG. 1, in accordance with an embodiment of the present disclosure; and

FIG. 4 illustrates a perspective view of a tanning aid comprising a tissue paper imprinted with a floral pattern, in accordance with an exemplary embodiment of the present disclosure.

Like reference numerals refer to like parts throughout the description of several views of the drawings.

### DETAILED DESCRIPTION OF THE DISCLOSURE

The exemplary embodiments described herein detail for illustrative purposes are subject to many variations in composition, structure, and design. It should be emphasized, however, that the present disclosure is not limited to a tanning aid and a kit as shown and described. It is understood that various omissions and substitutions of equivalents are contemplated as circumstances may suggest or render expedient, but these are intended to cover the application or implementation without departing from the spirit or scope of the claims of the present disclosure. Also, it is to be understood that the phraseology and terminology used herein is for the purpose of description and should not be regarded as limiting.

The terms “a” and “an” herein do not denote a limitation of quantity, but rather denote the presence of at least one of the referenced item.

The present disclosure provides a tanning aid and a kit for use in tanning a human body surface. The tanning aid comprises a tissue paper and a tanning composition. Utilization of the tanning aid, i.e. the tissue paper impregnated with the tanning composition, for wiping the human body surface enables application of the tanning composition on the human body surface evenly and precludes clumps on the human body surface. Further, the tanning aid enables application of the tanning composition on the human body surface in a timely and less cumbersome manner. Furthermore, the tanning aid dispenser configured to store the plurality of tanning aids precludes the need to carry bottled tanning composition, thereby precluding mess caused by leakage in such bottles.

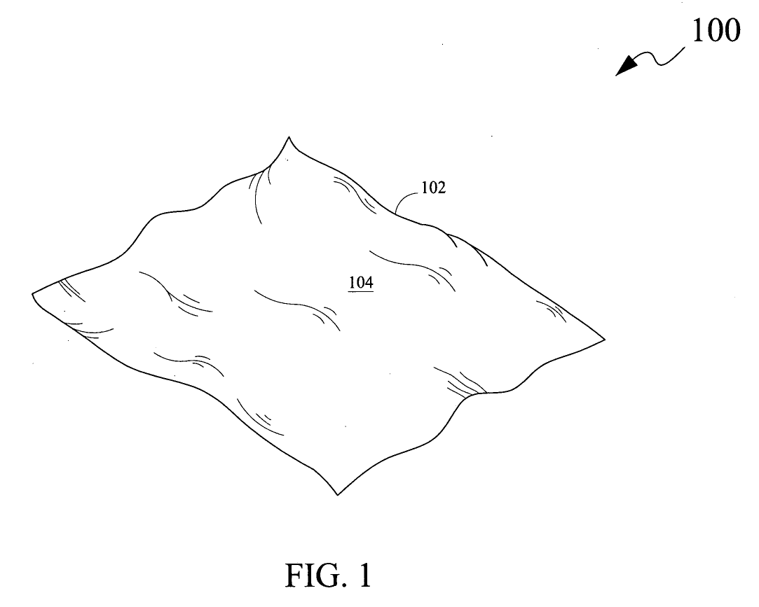

Referring now to FIG. 1, a perspective view of a tanning aid 100 is illustrated in accordance with an embodiment of the present disclosure. The tanning aid 100 includes a tissue paper 102 and a tanning composition (not shown). The tanning composition is impregnated in the tissue paper 102. The tanning composition includes an effective amount of at least one of a tanning lotion, a tanning oil and a bronzing agent.

The term ‘effective amount’, as described herein, may refer to an amount of the tanning composition that may be impregnated into the tissue paper 102 precluding any dripping of the tanning composition, and that enables a sufficient quantity of the tanning composition to be transferred when the tissue paper 102 is wiped on a human body surface (not shown and hereinafter referred to as a body surface) for effective application of the tanning composition to the body surface. Further, the effective amount of at least one of the tanning lotion, the tanning oil or the bronzing agent impregnated in the tissue paper 102, is sufficient to form a layer on the body surface for facilitating the tanning of the body surface and protecting the body surface against harmful ultraviolet (UV) radiation of sun or a tanning bed.

In an embodiment of the present disclosure, the tanning lotion may include at least one of melanin, L-Tyrosine, tea oil, green tea extract, hempseed oil and copper, as an ingredient. In an embodiment of the present disclosure, the tanning oil may include at least one of grape seed oil, coconut oil and sesame oil. An example of the bronzing agent may be dihydroxyacetone (DHA). It will be evident to a person skilled in the art that the ingredients and examples of the tanning lotion, the tanning oil and the bronzing agent are disclosed for exemplary purposes and as such may not be considered as limiting. Further, the bronzing agent may be combined with other ingredients, such as the tanning lotion or the tanning oil, for facilitating the tanning of the body surface, and also for protecting the body surface against the harsh UV rays of the sun or the tanning bed.

The tissue paper 102 impregnated with the tanning composition, is utilized for wiping the body surface for applying the tanning composition on the body surface. In an embodiment of the present disclosure, impregnation of the tanning composition into the tissue paper 102 may be carried out by soaking the tissue paper 102 in the tanning composition for a time period sufficient to allow absorption of the tanning composition into the tissue paper 102. However, it will be evident to a person skilled in the art that impregnation of the tissue paper 102 with the tanning composition by soaking the tissue paper 102 in the tanning composition is disclosed herein for exemplary purposes only, and, as such may not be considered as limiting. A suitable humectant, such as glycerin, may be added to the tanning composition to retain moisture in the tissue paper 102 for ensuring longer retention of the tanning composition in the tissue paper 102 after impregnation.

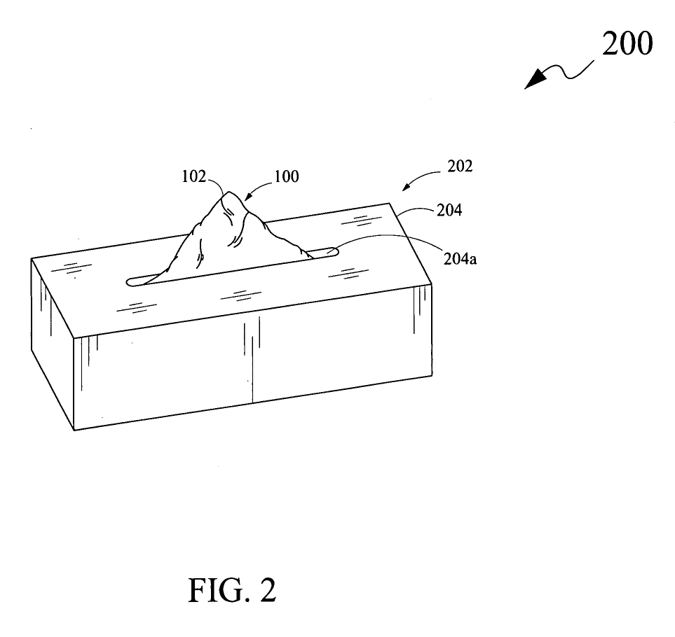

The tissue paper 102 of the FIG. 1 may be a standard tissue paper or even a sanitary wipe made from a biodegradable material such as non-woven fabric. Such a biodegradable tissue paper 102 is lightweight and decomposes easily, thereby making the tissue paper 102 environmentally friendly. In an embodiment of the present disclosure, the tissue paper 102 may be 9 inches in the length dimension and 11 inches in the width dimension. However, it will be evident to those skilled in the art that the length dimension and the width dimension are disclosed for exemplary purposes and as such may not be considered as limiting. In an embodiment of the present disclosure, various logos and stylized patterns may be imprinted on a surface 104 of the tissue paper 102 to make the tissue paper 102 attractive in appearance. Moreover, the tissue paper 102 may be designed to include different colors to suit the user's preference. To enhance a portability of the tanning aid 100, a kit including a tanning aid dispenser may be provided as depicted in conjunction with FIG. 2.

Now referring to FIG. 2, a perspective view of a kit 200 for use in tanning of a body surface (not shown) is disclosed, in accordance with an embodiment of the present disclosure. The kit 200 comprises a plurality of tanning aids (not shown) and a tanning aid dispenser 202. The tanning aid dispenser 202 is configured to store the plurality of tanning aids. The tanning aid dispenser 202 is capable of dispensing a tanning aid, such as the tanning aid 100 of FIG. 1, of the plurality of tanning aids.

In FIG. 2, the tanning aid dispenser 202 is depicted to include a box-shaped housing 204 with an opening 204a through which a portion of the tanning aid 100 extends in an outwardly direction. A user may retrieve the tanning aid 100 by applying a pulling force on the portion of the tanning aid 100 extending outwardly. It will be evident to a person skilled in the art that the plurality of tanning aids may be stored in the tanning aid dispenser 202 in a manner such that a portion of another tanning aid of the plurality of tanning aids may extend outwardly from the tanning aid dispenser 202 on retrieval of the tanning aid 100 from the tanning aid dispenser 202.

Each tanning aid of the plurality of tanning aids includes a tissue paper, such as the tissue paper 102, and a tanning composition. The tanning composition is impregnated in the tissue paper 102. As explained in conjunction with FIG. 1, the tanning composition includes an effective amount of at least one of a tanning lotion, a tanning oil and a bronzing agent. Further, as explained in conjunction with FIG. 1, the tanning lotion may include at least one of melanin, L-Tyrosine, tea oil, green tea extract, hempseed oil and copper, as ingredient; the tanning oil may include at least one of grape seed oil, coconut oil and sesame oil; and the bronzing agent may be dihydroxyacetone (DHA).

In FIG. 2, a portion of the tanning aid 100, i.e., a tissue paper 102 impregnated with the tanning composition is depicted to extend outwardly from the opening 204a. The portion of the tissue paper 102 extending outwardly may be grabbed and pulled by the user for retrieving the tanning aid 100. The tanning aid 100, i.e., the tissue paper 102 impregnated with the tanning composition, is utilized for wiping the body surface for applying the tanning composition on the body surface. Wiping the body surface with the tissue paper 102 impregnated with the tanning composition applies, i.e., covers the body surface with a layer of the tanning composition. Covering the body surface with the layer of the tanning composition facilitates a tanning of the body surface, and, moreover protects the body surface from the harsh UV rays of the sun or the tanning bed.

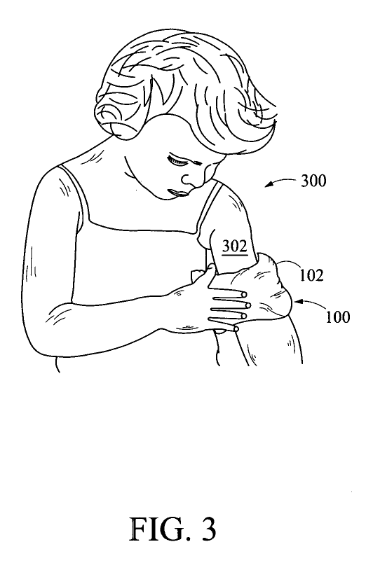

The tanning aid dispenser 202 may be dimensioned to include sizes suitable for carrying a sufficient number of tanning aids impregnated with the tanning composition, in a travel bag and the like, thereby, eliminating the need for carrying the tanning composition in separate bottled containers while heading outdoors for tanning under the sun or while heading to a tanning bed center. The box-shaped structure 204 of the tanning aid dispenser 202 may be composed of a lightweight plastic material. However, it will be evident to a person skilled in the art to use any suitable material for configuring the box-shaped structure of the tanning aid dispenser 202. The application of the tanning aid 100 to the body surface is depicted in FIG. 3.

Now referring to FIG. 3, a user 300 wiping her body surface 302 with the tanning aid 100 is illustrated, in accordance with an embodiment of the present disclosure. As explained in conjunction with FIGS. 1 and 2, the user 300 may carry the kit 200 (depicted in FIG. 2) in a handbag or a travel bag while heading outdoors for tanning under the sun or heading to the tanning bed center, and, thereafter the user 300 may retrieve the tanning aid 100 from the tanning aid dispenser 202. On retrieving the tanning aid 100 from the tanning aid dispenser 202 (depicted in FIG. 2), the user 300 may utilize the tissue paper 102 impregnated with the tanning composition for wiping the body surface 302.

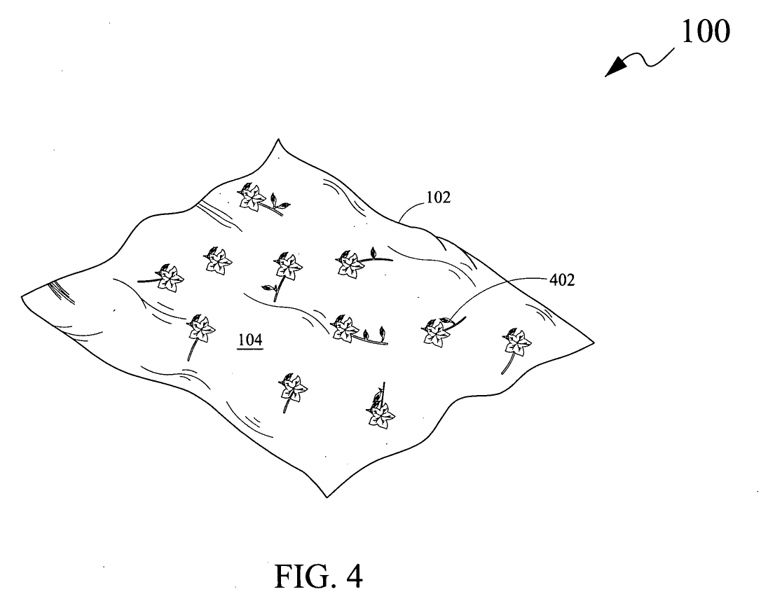

Wiping the body surface 302 with the tissue paper 102 impregnated with the tanning composition covers the body surface 302 with a layer of the tanning composition. Covering the body surface 302 with the layer of the tanning composition facilitates a tanning of the body surface 302, and, moreover protects the body surface 302 from the harsh UV rays of the sun or the tanning bed. It will be evident to a person skilled in the art that the user 300 as depicted in FIG. 3, is illustrated for exemplary purposes, and that the tanning aid 100 may be utilized by men, women and children with different complexion types. Various logos and stylized patterns may be imprinted on the surface 104 of the tissue paper 102 to make the tissue paper 102 attractive in appearance to the user 300. One such pattern is illustrated in FIG. 4.

FIG. 4 illustrates a perspective view of the tanning aid 100 including the tissue paper 102 imprinted with a floral pattern 402 on the surface 104, in accordance with an exemplary embodiment of the present disclosure. The floral pattern 402 is designed for increasing an aesthetic appearance of the tanning aid 100 and thereby better appeal to a user, such as the user 300. It will be evident to those skilled in the art that the floral pattern 402 depicted in FIG. 4, is illustrated for exemplary purposes only and that various logos or designs may be imprinted on the surface 104 of the tissue paper 102 for making the tanning aid 100 attractive in appearance to the user.

Various embodiments of the present disclosure offer following advantages. A tanning aid, such as the tanning aid 100 may be utilized for wiping a human body surface, such as the body surface 302, for applying the tanning composition to the body surface. The utilization of the tanning aid, i.e. the tissue paper impregnated with the tanning composition, for wiping the body surface enables application of the tanning composition on the body surface evenly and precludes clumps on the body surface. Further, the tanning aid enables application of the tanning composition on the body surface in a timely and less cumbersome manner. Furthermore, the tanning aid dispenser configured to store the plurality of tanning aids precludes the need to carry bottled tanning composition, thereby precluding messes caused by leakage in such bottles.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present disclosure.

What is claimed is:

1. A tanning aid, the tanning aid comprising:
a tissue paper; and
a tanning composition comprising an effective amount of at least one of a tanning lotion, a tanning oil and a bronzing agent, the tanning composition impregnated in the tissue paper,
wherein a human body surface is wiped with the tissue paper impregnated with the tanning composition for applying the tanning composition to the human body surface.

2. The tanning aid of claim 1, wherein the tissue paper is composed of biodegradable material.

3. The tanning aid of claim 1, wherein the tanning lotion comprises at least one of melanin, L-Tyrosine, tea oil, green tea extract, hempseed oil and copper.

4. The tanning aid of claim 1, wherein the tanning oil comprises at least one of grape seed oil, coconut oil and sesame oil.

5. The tanning aid of claim 1, wherein the bronzing agent is dihydroxyacetone.

6. A kit for use in tanning of a human body surface, the kit comprising:
a plurality of tanning aids, each tanning aid of the plurality of tanning aids comprising
a tissue paper, and
a tanning composition comprising an effective amount of at least one of a tanning lotion, a tanning oil and a bronzing agent, the tanning composition impregnated in the tissue paper; and
a tanning aid dispenser configured to store the plurality of tanning aids, wherein the tanning aid dispenser is capable of dispensing a tanning aid of the plurality of tanning aids, and,
wherein the human body surface wiped with the tissue paper impregnated with the tanning composition applies the tanning composition to the human body surface.

7. The kit of claim 6, wherein the tissue paper is composed of biodegradable material.

8. The kit of claim 6, wherein the tanning lotion comprises at least one of melanin, L-Tyrosine, tea oil, green tea extract, hempseed oil and copper.

9. The kit of claim 6, wherein the tanning oil comprises at least one of grape seed oil, coconut oil and sesame oil.

10. The kit of claim 6, wherein the bronzing agent is dihydroxyacetone.
